(19) United States
(12) Patent Application Publication
(10) Pub. No.: US 2012/0322988 A1
(43) Pub. Date: Dec. 20, 2012

(54) THERAPEUTIC AGENT FOR USE IN A METHOD OF TREATING PSORIASIS OR ATOPIC DERMATITIS

(76) Inventor: Masayoshi Shichiri, Tokyo (JP)

(21) Appl. No.: 13/509,150

(22) PCT Filed: Nov. 12, 2010
(86) PCT No.: PCT/JP2010/070627

§ 371 Date: Aug. 30, 2012

(30) Foreign Application Priority Data
Nov. 13, 2009 (JP) 2009-260031

(51) Int. Cl. C07K 16/12; C07K 1/00

(52) U.S. Cl. 530/389.5; 424/165.1

(57) ABSTRACT

The present invention provides a therapeutic agent for psoriasis or atopic dermatitis, and comprises anti-Staphylococcus aureus antibodies as the active ingredient. A therapeutic agent for psoriasis or atopic dermatitis is specifically provided. This agent comprises comprehensive anti-S. aureus surface antibodies, including antibodies against entire cell-surface proteins, as the active ingredient. The agent is obtainable by: (a) treating S. aureus with a protein crosslinking/fixation reagent to fix proteins expressed on the surface of S. aureus via intramolecular crosslinking; (b) administering S. aureus treated with the protein crosslinking/fixation reagent for protein fixation to an animal as an immunogen; and (c) obtaining an antibody from the animal.

### TECHNICAL FIELD

The present invention relates to a therapeutic agent for use in a method of treating psoriasis or atopic dermatitis that comprises an antibody as the active ingredient.

### BACKGROUND ART

The pathogeneses of psoriasis and of atopic dermatitis remain to be elucidated, but both are categorized as allergic skin diseases. Although complex interactions among a variety of genetic/environmental factors are known to play central roles in the development of atopic dermatitis, exact pathogenic mechanisms remain largely unknown (Non-Patent Literature 1). Many factors have been studied in search of possible triggers/inducers involved in the initiation or progression of the diseases including bacteria such as Staphylococcus aureus, a resident bacterium on the human skin. However, it is well established that such bacteria do not cause or aggravate atopic dermatitis. The pathogenic mechanism of psoriasis is less well understood, rendering its proper treatment unfeasible (Non-Patent Literature 2). Therefore, adrenocortical steroid hormones that act against allergic mechanisms while promoting bacterial proliferation are currently used as therapeutic agents.

S. aureus, a facultative anaerobic Gram-positive coccus present on human skin, pores, and in the nasal cavity, may enter the human body through wounds, and causes a variety of suppurative diseases, pneumonia, sepsis, meningitis, and others. Additionally, S. aureus toxin is known to cause food poisoning and toxic shock syndrome.

Numerous proteins are expressed by bacteria and viruses. Potentially beneficial monoclonal antibodies have been raised against specific antigen proteins and assayed to determine their application in medical therapy and related fields. However, specific recognition and targeting of whole cells using an antibody against a single antigen molecule expressed on cell surface has limitations.

Antibiotics have been primarily used to prevent or treat infectious diseases caused by bacteria and certain other organisms. Attenuated virus vaccines have also been administered to allow production of antiviral antibodies to prevent or treat viral infectious diseases. However, selection of appropriate antibiotics poses a risk of subsequent emergence of drug-resistant strains, while the issue of vaccine safety limits the spectrum of applicable infectious diseases.

### CITATION LIST

### Non-Patent Literature

Non-Patent Literature 1: Bieber T, Novak N: Pathogenesis of atopic dermatitis: new developments, Current Allergy and Asthma Reports, 2009, 9 291-294
Non-Patent Literature 2: Wippel-Slupetzky, K. Stingl, G: Future perspectives in the treatment of psoriasis, Current Problems in Dermatology, 2009, 38:172-89

### SUMMARY OF THE INVENTION

The present invention provides a therapeutic agent for psoriasis and atopic dermatitis, with anti-S. aureus antibodies, as the active ingredient. These comprehensive antibodies are specifically raised against entire proteins expressed on the surface of S. aureus.

The present inventor assumed that psoriasis and atopic dermatitis represent skin lesions induced by a skin-resident bacterium, S. aureus, and/or by toxins produced by S. aureus at erosive scratched skin sites. The present inventors raised anti-S. aureus antibodies by the method for producing comprehensive anti-surface antibodies using a microorganism treated with a protein crosslinking/fixation reagent as an antigen (JP Patent Application No. 2008-324257), and conducted intensive studies to elucidate whether such anti-S. aureus antibodies could treat psoriasis and atopic dermatitis.

First, the present inventor hypothesized that a toxin produced by S. aureus could be an aggravating factor of psoriasis and atopic dermatitis, based on the recognition that the toxin is a superantigen that activates T cells in peripheral blood.

The present inventor used S. aureus cells that had been surface-treated with formaldehyde for protein fixation to immunize animals as antigens, and raised comprehensive polyclonal antibodies against entire antigen protein molecules that had been fixed on the surface of S. aureus.

Repeated application of the anti-S. aureus surface antibodies to lesions resulted in complete elimination of S. aureus from the lesions, enabling the treatment of psoriasis and atopic dermatitis. This completed the present invention.

Specifically, the present invention is described below.

[1] A therapeutic agent for psoriasis or atopic dermatitis, which comprises anti-S. aureus antibodies as the active ingredient.
[2] The therapeutic agent for psoriasis or atopic dermatitis according to [1], wherein the anti-S. aureus antibodies are comprehensive anti-S. aureus surface antibodies raised against entire protein molecules expressed on the surface of S. aureus, which is obtainable by: (a) treating S. aureus with a protein crosslinking/fixation reagent to fix proteins expressed on the surface of S. aureus via intramolecular crosslinking; (b) administering S. aureus treated with the protein crosslinking/fixation reagent for protein fixation to an animal as an immunogen, and (c) obtaining an antibody from the animal.
[3] The therapeutic agent for psoriasis or atopic dermatitis according to [2], wherein the protein crosslinking/fixation reagent is selected from the group consisting of formaldehyde, paraformaldehyde, and glutaraldehyde.
[4] The therapeutic agent for psoriasis or atopic dermatitis according to [2], wherein the protein crosslinking/fixation reagent is 1-38% (v/v) formalin.
[5] The therapeutic agent for psoriasis or atopic dermatitis according to any one of [2] to [4], wherein protein fixation is carried out using a protein crosslinking/fixation reagent for 10 minutes to 48 hours.
[6] The therapeutic agent for psoriasis or atopic dermatitis according to any one of [2] to [5], wherein an animal to which an immunogen is administered is a chicken.
[7] The therapeutic agent for psoriasis or atopic dermatitis according to [6], wherein antibodies are obtained from an egg laid by an immunogen-administered chicken.

This description includes part or all of the contents as disclosed in the description and/or drawings of Japanese Patent Application No. 2009-260031, which is a priority document of the present application.

### BRIEF DESCRIPTION OF DRAWINGS

FIG. 1 shows photos indicating the effect of the therapeutic agent of the present invention on atopic dermatitis patients (I).

FIG. 2 shows photos indicating the effect of the therapeutic agent of the present invention on atopic dermatitis patients (II).

FIG. 3 shows photos indicating the effect of the therapeutic agent of the present invention on atopic dermatitis-affected dogs (I).

FIG. 4 shows photos indicating the effect of the therapeutic agent of the present invention on atopic dermatitis-affected dogs (II).

FIG. 5 shows photos indicating the effect of the therapeutic agent of the present invention on atopic dermatitis-affected dogs (III).

FIG. 6 shows photos indicating the effect of the therapeutic agent of the present invention on psoriasis patients.

### DESCRIPTION OF EMBODIMENTS

The present invention is described in detail below.

The therapeutic agent for psoriasis and atopic dermatitis of the present invention comprises anti-S. aureus antibodies as the active ingredient. The anti-S. aureus antibodies can be produced using the whole or a part of a S. aureus cell via a conventionally known immunization method. The anti-S. aureus antibodies of the present invention may be either polyclonal or monoclonal. The anti-S. aureus antibodies are not restricted to complete antibodies, but could be functional fragments of antibodies. The term “functional fragment of an antibody” refers to a part of an antibody (i.e., partial fragment) maintaining at least one action of the antibody against the corresponding antigen. Specific examples of functional fragments include F(ab′)₂, Fab′, Fab, Fv, disulfide bond Fv, single chain Fv (scFv), and polymers thereof (D. J. King., Applications and Engineering of Monoclonal Antibodies, 1998, T. J. International Ltd.).

In addition, monoclonal antibodies used herein may be of one type. Alternatively, two or more types of monoclonal antibodies that recognize different epitopes (e.g., 2, 3, 4, or 5 types of monoclonal antibodies) may be used. Furthermore, the anti-S. aureus antibodies of the present invention also include recombinant antibodies that have been artificially modified to reduce heterologous antigenicity to humans. Examples of such antibodies include a chimeric antibody, a humanized antibody, and a human antibody, each of which can be produced by a conventional method.

Polyclonal antibodies comprising comprehensive cell surface antibodies are preferential, as described below.

Comprehensive surface antibodies are raised by directly using S. aureus cells as antigens. The S. aureus cell surfaces are treated with formaldehyde for protein fixation. By doing so, comprehensive antibodies against S. aureus can be produced. Here, the production of comprehensive antibodies against S. aureus refers to raising comprehensive antibodies against entire antigen protein molecules that are expressed on the surface of S. aureus. The obtained antibody is termed herein as comprehensive anti-S. aureus surface antibody. Comprehensive anti-S. aureus surface antibodies are polyclonal and comprise individual antibodies against many antigen proteins expressed on the surface of S. aureus. According to the method of the present invention, comprehensive anti-S. aureus surface antibodies, comprising antibodies against surface protein molecules, can be obtained by administering fixed S. aureus to animals. Here, the comprehensive anti-S. aureus surface antibodies do not necessarily comprise antibodies against all proteins expressed on the surface of S. aureus. However, they desirably comprise antibodies against most highly-abundant surface proteins.

The comprehensive anti-S. aureus antibodies can be produced by the method described below.

First, S. aureus is treated with an intramolecular protein crosslinking/fixation reagent for protein fixation. Examples of an intramolecular protein crosslinking/fixation reagent include aldehydes such as formaldehyde, paraformaldehyde, and glutaraldehyde. Preferably, fixation is carried out using formalin, which is a 35-38% (preferably 37%) aqueous formaldehyde solution.

When such an intramolecular protein crosslinking/fixation reagent is used as a fixation reagent, the reagent permeates S. aureus cells and then aldehyde in the reagent binds to an amino group (α-amino or ε-amino of a lysine residue) or a thiol group of a protein present on the surface of each S. aureus cell. This forms an intramolecular crosslink, resulting in protein coagulation/denaturation. Such crosslinking causes destruction of protein conformation, inhibiting various bioactivities related to enzyme activity, transport, secretion, and the like. Since the intramolecular protein crosslinking/fixation reagent induces a protein crosslinking reaction, it does not fix other substances such as lipids, but does allow leakage of these substances.

If a commercially available 35-38% aqueous formaldehyde solution (i.e., formalin) is used, fixation can be carried out using 1-38% (v/v) formalin; 2-20% (v/v) is preferable, and 5-10% (v/v) formalin is optimal. Here, formalin may be prepared using distilled water, physiological saline, buffer, or similar.

S. aureus can be homogenized and 5-20 mL formalin should be added per 1 g of pellets. For fixation, formalin is added to the S. aureus cell pellet, mixed, and then incubated at 4-30° C. for 30 minutes to 48 hours or longer. Viable S. aureus cells can be inactivated by short-time formalin treatment. However, according to the method of the present invention, inactivated S. aureus alone is not sufficient to be used as an antigen; adequate formalin treatment is required to induce intramolecular crosslinking of protein molecules expressed on the surface of S. aureus.

The fixed S. aureus pellet may be diluted with physiological saline or buffer, resuspended, and mixed with a known adjuvant as required, and administered as an immunogen in an animal model. Examples of such animals include mammals (such as mice, rats, nutrias, rabbits, sheep, goats, horses, and cattle) and birds (such as chickens and ostriches). Of these animals, birds such as chickens are preferable, because eggs containing IgY antibodies can be obtained.

Animals can be immunized with the immunogen by a conventional method. Such methods generally use intraperitoneal or subcutaneous injection of the immunogen. Immunization may be performed once, but preferably several times every 2-10 days. A booster may be given every 5-10 days, several times in total, after initial administration.

A method for preparing a S. aureus immunogen is described below. This method is an example, and thus the present invention is not limited thereto.

S. aureus cells are collected by centrifugation, and resuspended pellets are added to formalin, mixed by vortex, incubated for 30 minutes, and filtered through coarse filter paper to remove residues. The filtrate is collected, centrifuged, and the resultant cell pellet is resuspended in phosphate buffer. The resuspended cells can then be used as an immunogen. The comprehensive anti-surface-antibody composite of the present invention can be recovered from serum of an animal immunized with the above immunogen. In the case of a chicken model, the antibodies can be obtained from laid eggs. Chicken eggs with a high antibody titer can be obtained approximately 3 months following the final immunization. Chicken egg yolk contains IgY, while the egg albumen contains IgA and IgM. The comprehensive antibody composite obtained from whole eggs contains IgY, IgA, and IgM, while that from egg yolk contains IgY. In preparing an antibody composite of the present invention using chicken eggs, either whole eggs or egg yolk alone can be used.

To recover the antibody, either whole egg or egg yolk alone is powderized. Powderization should preferably be performed at a temperature of less than 60° C., because heat-labile antibody proteins lose their activity at 70° C. or higher. Eggs are first dehydrated by lyophilization, spray drying, or hot-air drying at 60° C. or below and, if necessary, blended until a very fine powder is obtained. This powder contains egg yolk oil components, and has grease and moisture. The oil components may be completely removed by defatting via an ultracold critical method.

Hereafter, the comprehensive anti-surface antibody composition obtained from chicken eggs is referred to as a “chicken egg antibody.”

The titer of the obtained antibodies can be determined by such methods as ELISA (enzyme-linked immunosorbent assay), EIA (enzyme immunoassay), RIA (radioimmunoassay), or immunofluorescence. The antibody titer may be determined using serum obtained from a subject animal. When using chicken eggs, the antibody titer of an extracted chicken egg may be determined.

The comprehensive antibodies against S. aureus produced by the present invention can be used to prevent or treat psoriasis and atopic dermatitis. Because the comprehensive anti-S. aureus antibodies of the present invention contain antibodies against entire proteins expressed on the surface of S. aureus, they can kill, eliminate, or remove S. aureus with strong antibacterial properties.

The prophylactic or therapeutic agent for psoriasis and atopic dermatitis, which comprises the comprehensive anti-S. aureus antibodies as the active ingredient, can be administered to animals. The antibody composite can be administered via oral, nasal, or non-enteral routes, in addition to intravenous, intramuscular, subcutaneous, or intraperitoneal injection. Alternatively, it can be applied locally to skin lesions as an ointment or liquid spray. Such ointment contains a carrier, such as fat, fatty oil, lanolin, Vaseline, paraffin, Plastibase®, wax, plaster, resin, plastic, glycols, fatty alcohols, glycerin, water or an emulsifier, and/or a suspending agent. The dose of the pharmaceutical composite of the present invention to be administered ranges from approximately 0.01-10000 mg daily, in a single or multiple dosing schedule, depending on symptoms, age, and weight.

The prophylactic or therapeutic antibody composite for psoriasis and atopic dermatitis may comprise a carrier, diluent, or excipient generally used in drug formulation. Examples of a carrier or excipient for tablets include lactose and magnesium stearate.

The therapeutic agent for psoriasis and atopic dermatitis of the present invention can be used for animals at risk of psoriasis or atopic dermatitis. Examples of such animals include humans, monkeys, and pet animals such as dogs and cats.

The therapeutic agent comprising anti-S. aureus antibodies of the present invention causes removal of S. aureus from psoriasis and atopic dermatitis lesions, making it possible to prevent aggravation of skin damage such as erosion of scratch wounds on skin surfaces due to S. aureus toxin.

Hereafter, embodiments of the present invention are described with reference to the following examples, although the technical scope of the present invention is not limited thereto.

### Example 1

### Production of Comprehensive Antibodies Against S. aureus as an Antigen

### (1) Antigen Preparation

S. aureus (MicroBiologics® Inc., purchased from Kanto Chemical Co., Ltd.) was cultured to prepare frozen S. aureus cell pellets. Approximately 3 mg of thawed S. aureus cell pellet was placed into 15-mL capped tubes containing 5 ml of 5% formalin solution, mixed several times using a vortex test tube mixer for approximately 10 minutes in total to obtain uniform solution, and then incubated for 30 minutes. Complete dissolution of the cell pellet in the 5% formalin solution resulted in fixation of S. aureus surface antigens.

The cell pellets dissolved in 5% formalin solution were centrifuged, and 1.5 mL of the bottom phase solution, containing the centrifuged sediment, was retrieved using a pipette.

Natural or suction filtration was conducted using coarse filter paper or a filter, followed by centrifugation. The supernatant was discarded to reduce the formalin concentration.

The sediment was dissolved in physiological saline. The resultant solution was used as an antigen for administration.

The antigen (1 mL) and white oil were mixed in a stepwise manner using an emulsion pump to prepare an emulsion (emulsified liquid). This emulsion was administered to animals.

### (2) Immunization

The emulsion (0.5 mL) prepared in (1) was administered subcutaneously to inguinal regions of 5-week-old female chickens. The antigen composite for administration was preheated to chicken body temperature (37° C.) to reduce discomfort to the chickens.

The same dose of the emulsified liquid was given as a booster to the chickens every 7 days, twice in total, following the initial administration.

Whole eggs laid by the immunized chickens were broken and stirred. A dried egg powder was produced using a GA22 spray dryer (Yamato Scientific Co., Ltd.), a hot air dryer, and a pulverizer. The spray dryer temperature was adjusted to 60° C. or lower.

ELISA was conducted to determine antibody titer.

### Example 2

### Treatment of Human Atopic Dermatitis

The therapeutic threshold for the chicken egg antibody produced by the method described in Example 1 was determined. The polyclonal antibody was mixed with petrolatum ointment with a final antibody composition of 1-5% of the formulation weight. These ointment formulations were applied to skin lesions of atopic dermatitis patients.

The threshold determination results showed that strong itching sensation and redness induced by S. aureus toxin (SA toxin) were reduced at each concentration.

A further study employed an ointment containing the polyclonal antibody mixed with petrolatum ointment at a weight ratio of 5%.

The ointment was administered to 10 atopic dermatitis patients, once or twice daily, and resulted in improvement in symptoms after approximately two weeks. It showed remarkable effects after two to three months.

Table 1 shows the results of the ointment effect evaluation test, including that 90% of the cases showed “Improved” or better results, demonstrating very high effectiveness.

TABLE 1
Ointment effect evaluation test results

| | | | Administration | |
|---|---|---|---|---|
| Subject | Sex | Age | period | Effect evaluation |
| A | ♂ | 35 | 6 months | (III) Very effective: |
| | | | (completed) | Photos attached |
| B | ♂ | 28 | 6 months | (I) Relatively improved: |
| | | | (continued) | Disappearance of the itching |
| | | | | sensation, attenuation of redness |
| C | ♂ | 32 | 3 months | (III) Very effective: Recovery |
| | | | (completed) | to normal skin |
| D | ♀ | 24 | 6 months | (III) Very effective: Recovery |
| | | | (completed) | to normal skin |
| E | ♂ | 8 | 1 month | (—) Not effective: No |
| | | | (completed) | improvement of symptoms |
| F | ♀ | 21 | 2 months | (II) Effective: Disappearance |
| | | | (continued) | of itching sensation and redness |
| G | ♂ | 40 | 8 months | (I) Relatively improved: |
| | | | (continued) | Poor compliance |
| H | ♀ | 34 | 3 months | (III) Very effective: Recovery |
| | | | (completed) | to normal skin |
| I | ♀ | 24 | 6 months | (III) Very effective: Recovery |
| | | | (completed) | to normal skin |
| J | ♀ | 31 | 4 months | (III) Very effective: Recovery |
| | | | (completed) | to normal skin |

Effect evaluation criteria
(III) Very effective: Disappearance of itching sensation and redness and recovery to normal skin
(II) Effective: Disappearance of both itching sensation and redness
(I) Relatively improved: Disappearance of itching sensation or redness
(—) Not effective: No exhibited effects

Ninety percent of cases showed scores of “Relatively improved” or better (efficacy rate). Among these, “Very effective” results accounted for 60% of cases, “Effective” results accounted for 10% of cases, and “Relatively improved” results accounted for 20% of cases. No effect was seen in 10% of cases.

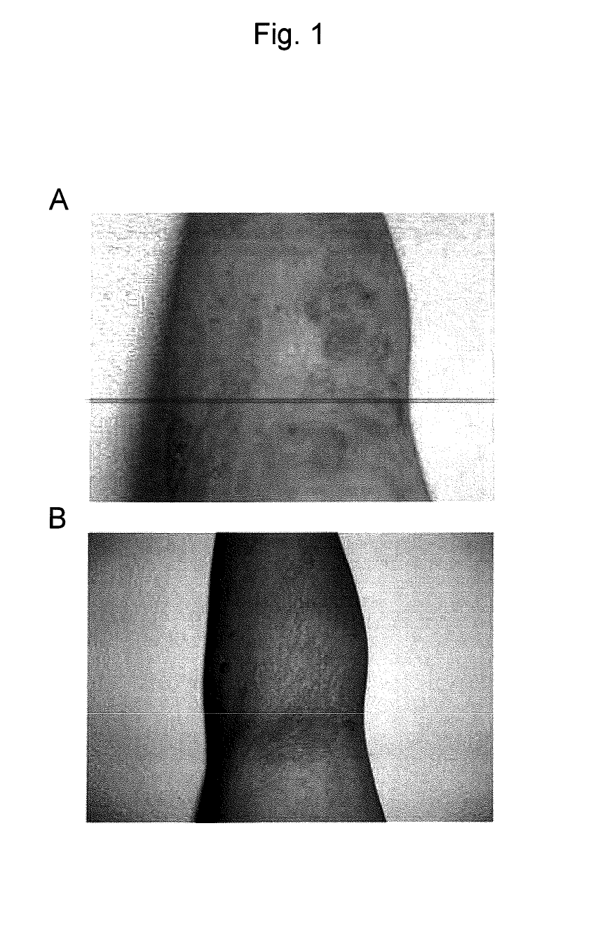

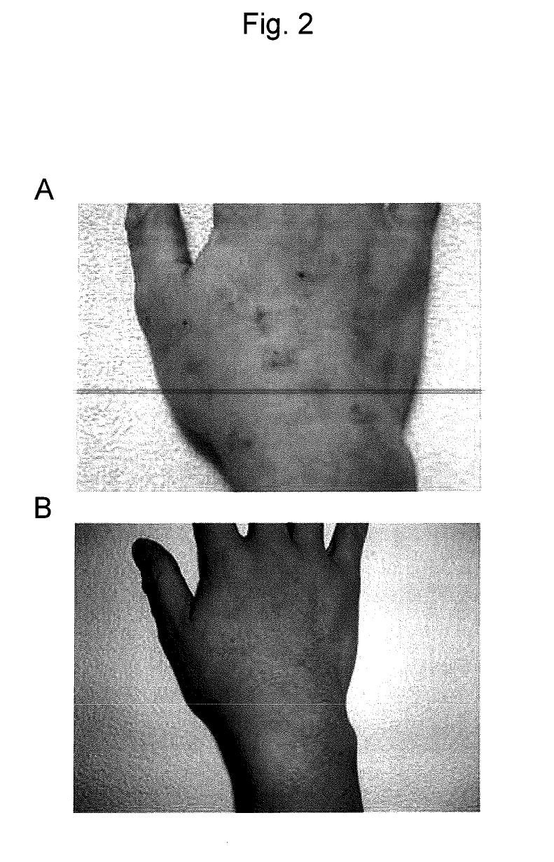

In addition, FIGS. 1 and 2 show photos indicating the effects of the therapeutic agent of the present invention upon atopic dermatitis patients. FIGS. 1A and 2A show lesions of atopic dermatitis patients prior to treatment with the therapeutic agent of the present invention. FIGS. 1B and 2B show lesions of atopic dermatitis patients following treatment with the therapeutic agent of the present invention. As shown in the figures, the use of the therapeutic agent of the present invention resulted in the disappearance or alleviation of symptoms in atopic dermatitis patients.

### Example 3

### Treatment of Atopic Dermatitis of Dogs

Dogs have a life expectancy of more than 10 years. Dogs aged five years and older have a high rate of developing atopic dermatitis. In this example, effects of the therapeutic agent of the present invention upon atopic dermatitis of dogs were evaluated.

Ointment was prepared by the method described in Example 2 using the anti-S. aureus antibody produced by the method described in Example 1. The ointment was applied to skin lesions in seven dogs with atopic dermatitis once daily during a series of consecutive days.

Table 2 shows the effect evaluation test results. As shown in Table 2, improvement of atopic dermatitis was confirmed in many cases.

TABLE 2
The effect evaluation test results of SA-antibody-
containing ointment for dog atopic dermatitis

| | | | Administration | |
|---|---|---|---|---|
| Case | Dog breed/Sex | Age | period | Effect evaluation |
| Case 1 | Long | 7 | 1 month | (III) Very effective: |
| | dachshund/♂ | | (completed) | Recovery to normal |
| | | | | skin |
| Case 2 | Miniature | 9 | 1.5 months | (III) Very effective: |
| | dachshund/♀ | | (completed) | Recovery to normal |
| | | | | skin |
| Case 3 | Miniature | 15 | 1.5 months | (II) Effective: |
| | dachshund/♂ | | (continued) | Disappearance of |
| | | | | itching sensation |
| | | | | and redness |
| Case 4 | Shih Tzu/♀ | 12 | 1.5 months | (—) No improvement |
| | | | (continued) | of symptoms |
| Case 5 | Pug/♀ | 11 | 1 month | (—) No improvement |
| | | | (continued) | of symptoms |
| Case 6 | Shiba inu/♀ | 10 | 1 month | (—) No improvement |
| | | | (continued) | of symptoms |
| Case 7 | Papillon/♂ | 2 | 1 month | (II) Effective: |
| | | | (continued) | Disappearance of |
| | | | | itching sensation |
| | | | | and redness |

Effect evaluation criteria
(III) Very effective: Disappearance of itching sensation or redness and recovery to normal skin
(II) Effective: Disappearance of itching sensation and redness
(I) Relatively improved: Disappearance of redness
(—) No improvement of symptoms

“Relatively improved” or better results were recorded in 57% of cases (effective rate). Among these, “Very effective” results accounted for 29% of cases, “Effective” results were seen in 29% of cases, with no “Relatively improved” cases. No improvement was seen in 42% of cases.

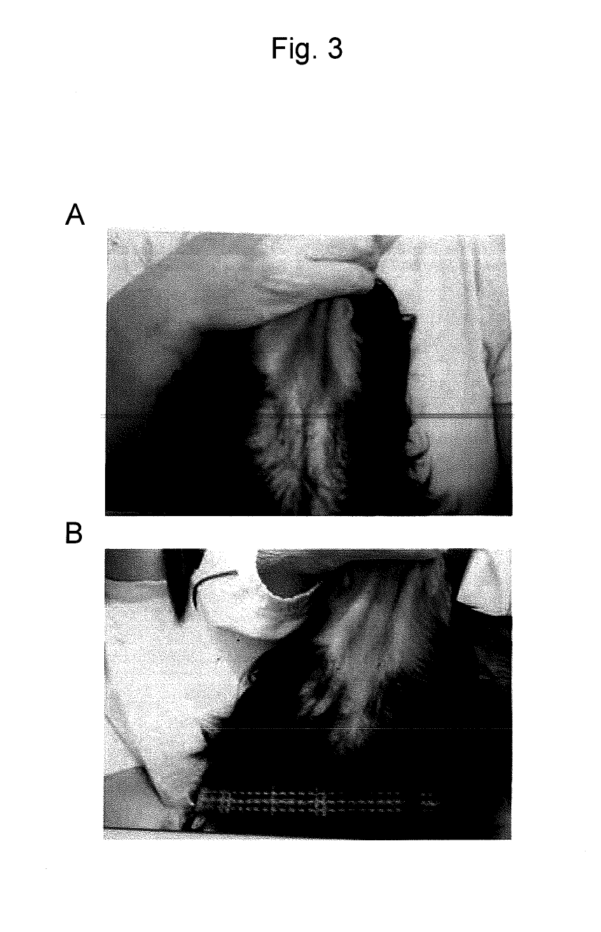

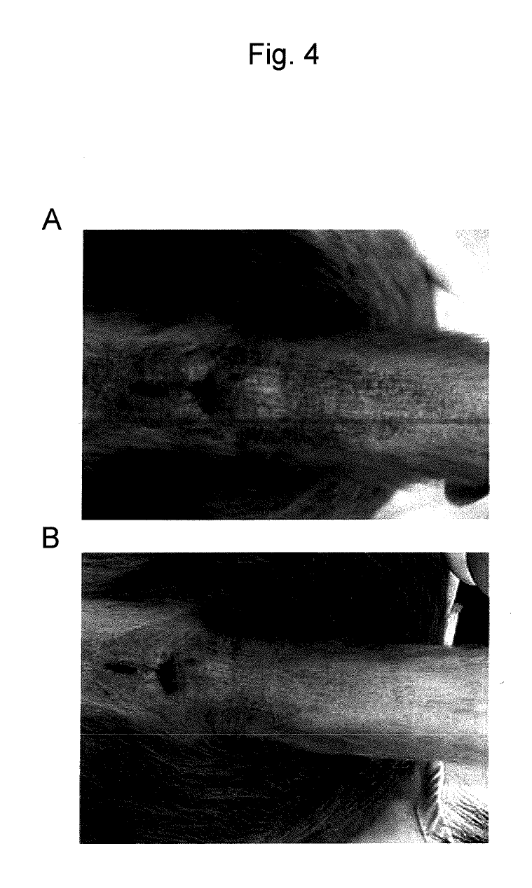

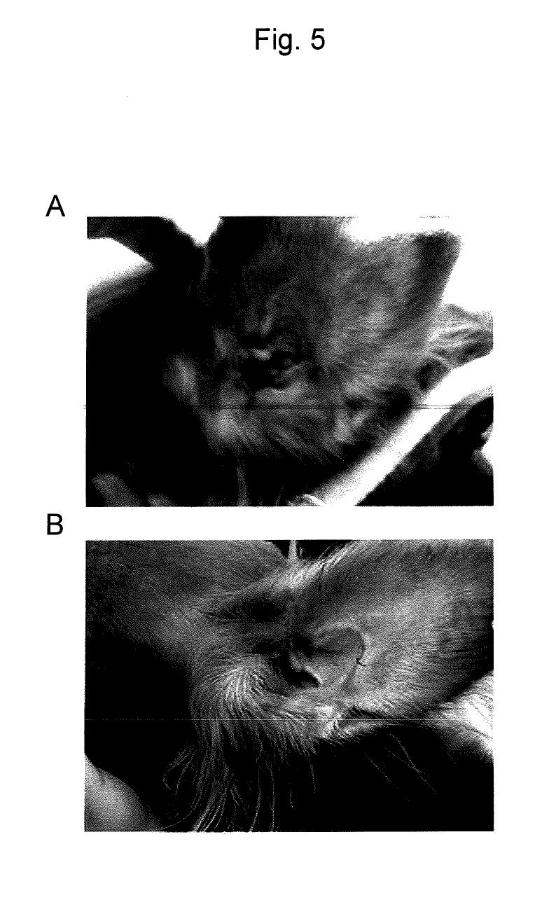

FIGS. 3 and 5 show photos indicating the effects in dogs affected with atopic dermatitis. FIGS. 3A, 4A, and 5A show lesions in dogs affected with atopic dermatitis prior to treatment with the therapeutic agent of the present invention, while FIGS. 3B, 4B, and 5B show lesions in the same dogs following treatment. As shown in the figures, the use of the therapeutic agent of the present invention resulted in disappearance or alleviation of symptoms in dogs affected with atopic dermatitis. In this example, the effects in dogs were relatively less than those observed in humans (Example 2). The onset of effects in dogs aged 10 and older appeared to be delayed.

### Example 4

### Treatment of Human Psoriasis

Ointment containing the anti-S. aureus antibody produced according to the method described in Example 1 was prepared by the method described in Example 2 and applied to lesions of psoriasis patients once or twice daily. The effects were confirmed in approximately two weeks and significant improvements were evident in two to three months. The lesions were monitored for a period following the initiation of treatment.

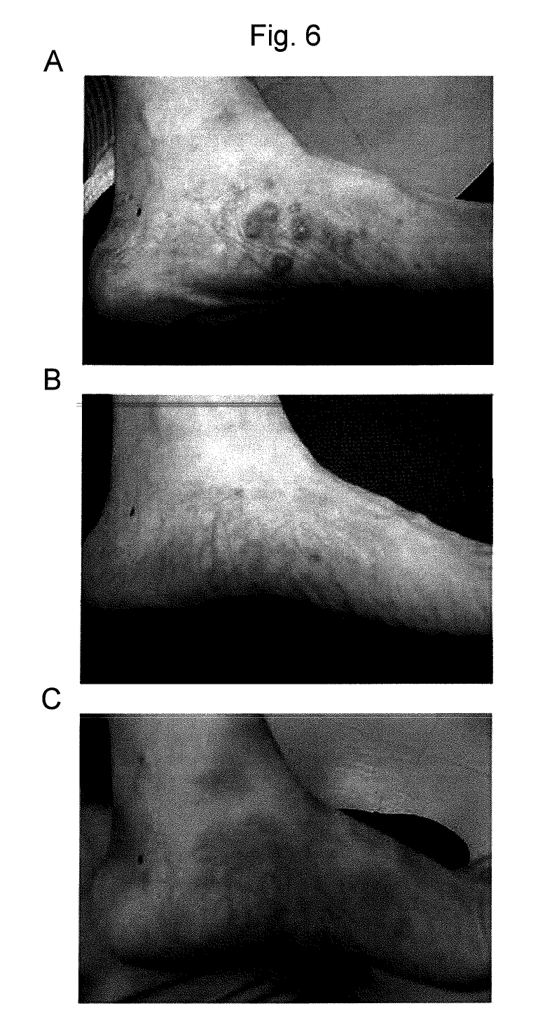

FIG. 6 shows photos indicating the effects on psoriasis patients. FIGS. 6A, 6B, and 6C show lesions on days 15, 30, and 90, respectively, following treatment initiation. As shown in the figures, continuous application resulted in alleviation of symptoms.

### INDUSTRIAL APPLICABILITY

Hitherto, no direct causal relationship between S. aureus and psoriasis/atopic dermatitis has been suggested. The therapeutic agent of the present invention for psoriasis and atopic dermatitis comprises anti-S. aureus antibodies as the active ingredient, eliminates S. aureus from skin lesions, and is applicable for treatment of psoriasis and atopic dermatitis. In particular, the comprehensive anti-S. aureus antibodies of the present invention are raised by administering proteins expressed on the surface of S. aureus that are fixed by intramolecular crosslinking with the use of an intramolecular protein crosslinking reagent to animals, thus comprising antibodies against the entire spectrum of proteins expressed on the surface of S. aureus. An antibody composite comprising such comprehensive antibodies has high specificity and sensitivity to S. aureus that has not been achieved by conventional antibody production methods. A pharmaceutical composite comprising the antibodies can be effectively used for treatment of psoriasis and atopic dermatitis.

The composition comprising the anti-S. aureus antibodies of the present invention as the active ingredient can be used for the treatment of psoriasis and atopic dermatitis.

All publications, patents, and patent applications cited herein are incorporated herein by reference in their entirety.

1. A therapeutic agent for psoriasis or atopic dermatitis, which comprises an anti-S. aureus antibodies as the active ingredient.

2. The therapeutic agent for psoriasis or atopic dermatitis according to claim 1, wherein the anti-S. aureus antibodies are comprehensive anti-S. aureus surface antibodies comprising antibodies against entire proteins expressed on the surface of Staphylococcus aureus, which is obtainable by: (a) treating S. aureus with a protein crosslinking/fixation reagent to fix proteins expressed on the surface of S. aureus via intramolecular crosslinking; (b) administering S. aureus treated with the protein crosslinking/fixation reagent for protein fixation to an animal as an immunogen; and (c) obtaining an antibody from the animal.

3. The therapeutic agent for psoriasis or atopic dermatitis according to claim 2, wherein the protein crosslinking/fixation reagent is selected from the group consisting of formaldehyde, paraformaldehyde, and glutaraldehyde.

4. The therapeutic agent for psoriasis or atopic dermatitis according to claim 2, wherein the protein crosslinking/fixation reagent is 1-38% (v/v) formalin.

5. The therapeutic agent for psoriasis or atopic dermatitis according to claim 2, wherein protein fixation is carried out using a protein crosslinking/fixation reagent for 10 minutes to 48 hours.

6. The therapeutic agent for psoriasis or atopic dermatitis according to claim 2, wherein an animal to which an immunogen administered is a chicken.

7. The therapeutic agent for psoriasis or atopic dermatitis according to claim 6, wherein antibodies are obtained from an egg laid by an immunogen-administered chicken.
